(19) United States
(12) Patent Application Publication
(10) Pub. No.: US 2016/0130260 A1
(43) Pub. Date: May 12, 2016

(54) TERT-BUTYL SULPHOXIDE METHOD FOR PRODUCING FESTINAVIR

(71) Applicant: BRISTOL-MYERS SQUIBB COMPANY, Princeton, NJ (US)

(72) Inventors: Adrian Ortiz, Hoboken, NJ (US); Tamas Benkovics, Somerset, NJ (US); Martin D. Eastgate, Titusville, NJ (US)

(21) Appl. No.: 14/896,995

(22) PCT Filed: Jun. 11, 2014
(86) PCT No.: PCT/US2014/041918

§ 371 Date: Dec. 9, 2015

Related U.S. Application Data
(60) Provisional application No. 61/834,480, filed on Jun. 13, 2013.

(51) Int. Cl. C07D 407/04

(52) U.S. Cl. CPC C07D 407/04

(57) ABSTRACT

Tert-butyl sulphoxide method for producing festinavir is set forth.

### CROSS REFERENCE TO RELATED APPLICATION

This application claims the benefit of U.S. Provisional Application Ser. No. 61/834,480 filed Jun. 13, 2013 which is herein incorporated by reference in its entirety.

### FIELD OF THE INVENTION

The present invention relates to a method for producing the compound festinavir. More particularly, the invention is directed to an improved method for producing festinavir in good yield utilizing a different starting material and reaction mechanism(s) than has been used to date. The invention is also directed to the intermediate compounds, as well as to the compound festinavir itself, which is produced by the process(es) herein.

### BACKGROUND OF THE INVENTION

The compound known as festinavir is a nucleoside reverse transcriptase inhibitor (NRTI) which is being developed for the treatment of HIV infection. The drug has shown considerable efficacy in early development, and with perhaps less toxicity than some other NRTIs, such as the drug stavudine (marketed under the trade name ZERIT®). Festinavir has the chemical formula C₁₁N₂O₄H₈, and the structural formula:

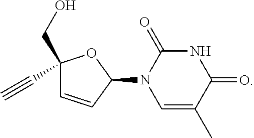 [SMILES: C#C[C@@]1(CO)C=C[C@H](n2cc(C)c(=O)[nH]c2=O)O1]

Festinavir was developed by Yale University in conjunction with two Japanese research scientists, and is protected by U.S. Pat. No. 7,589,078, the contents of which are incorporated herein by reference. The '078 patent sets forth the synthesis of the primary compound, and other structural analogs. In addition, Oncolys BioPharma, Inc. of Japan has now published US 2010/0280235 for the production of 4′ ethynyl D4T. As starting raw material, the Oncolys method utilizes a substituted furan compound, furfuryl alcohol. In another publication by Nissan Chemical Industries of Japan, and set forth in WO 2011/099443, there is disclosed a method for producing a beta-dihydrofuran deriving compound or a beta-tetrahydrofuran deriving compound. In this process, a diol compound is used as the starting material. Nissan has also published WO 2011/09442 directed to a process for the preparation of a β-glycoside compound. Two further publications, each to Hamari Chemicals of Japan, WO 2009/119785 and WO 2009/125841, set forth methods for producing and purifying ethynyl thymide compounds. Pharmaset, Inc. of the U.S. has also published US 2009/0318380, WO 2009/005674 and WO 2007/038507 for the production of 4′-nucleoside analogs for treating HIV infection. Also noted are two patent applications to Bristol-Myers Squibb, PCT/US14/33972 filed Apr. 14, 2014 entitled “5-Methyluridine Method of Producing Festinavir” and WO 2013/177243 entitled “Sulfilimine and Sulphoxide Methods for Producing Festinavir”.

What is now needed in the art are new methods for the production of festinavir. The newly developed methods should be cost effective and obtain the final compound in relatively high yield, and should also utilize different starting material(s) and process mechanisms than what has been set forth in the available art, or is otherwise available to the skilled artisan.

### SUMMARY OF THE INVENTION

In a first embodiment, the invention is directed to a process for making the compound of Formula I:

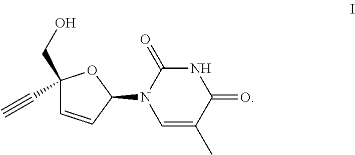 [SMILES: C#C[C@@]1(CO)C=C[C@H](n2cc(C)c(=O)[nH]c2=O)O1]

which comprises:
(1) contacting the starting compound, (S)—S-tert-butyl 2-methylpropane-2-sulfinothioate with the Grignard reagent generated from 3-bromo-1,1-dimethoxypropane to produce compound 1a:

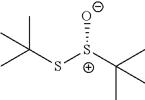

(S)—S-tert-butyl 2-methylpropane-2-sulfinothioate

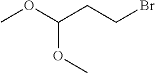

3-bromo-1,1-dimethoxypropane

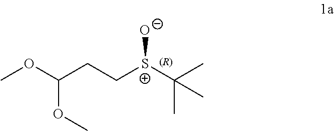

and then contacting compound 1a with p-thiocresol and BF₃-Et₂O to produce compound 1b; and then contacting compound 1b

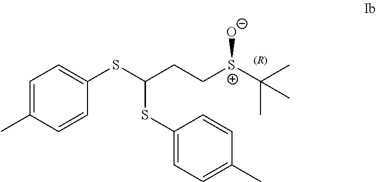

where X is sulfur is preferred
where R′ is aryl is preferred

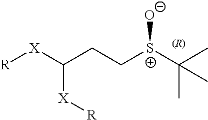

### X=S,O

R′=alkyl, cycloalkyl,
with n-BuLi and compound 2 to produce the compound 3, after removal of the silicon groups with TBAF and then re-protection of the C-5 hydroxy group as its benzoate ester (4-DMAP, Bz₂O):

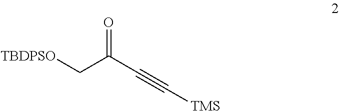

where R″=TBDPS is preferred
Where R′″=TMS is preferred

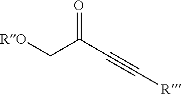

R″=H, alkyl, benzyl
allyl, alkyl ester
aryl ester, or SiY₃

### R′″=H, SiY₃

Y=aryl or alkyl

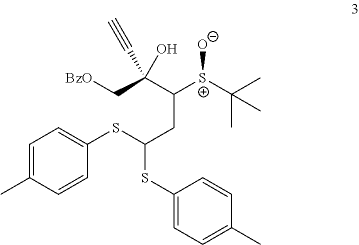

where R′=aryl is preferred
where R″=Bz is preferred
where X=sulfur is preferred

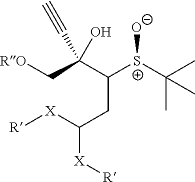

### X=O,S

R′=alkyl, cycloalkyl, aryl
R″=H, alkyl, benzyl, allyl, aryl, alkyl, ester, aryl ester, or SiY₃
Y=alkyl or aryl
; and then contacting the compound 3 with Bis-TMS-thymine

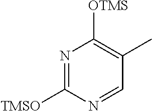

and NBS in nitromethane or CH₃CN to produce compound 4; and

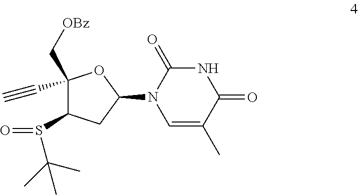

Where R″=Bz is preferred

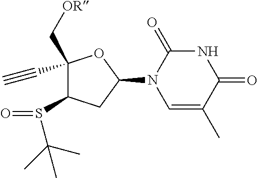

R″=SiY₃, alkyl ester, aryl ester, benzyl ether, allyl ether.
Y=ary or alkyl
; and contacting the compound 4 with propiolic acid with n-BuOH or t-Amyl-OH as solvent and heating to produce compound 5:

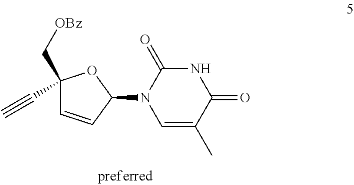

When R″=benzoate is preferred

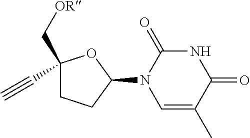

R′=SiY₃, alkyl ester, aryl ester, benzyl ether, allyl ether.
Y=aryl or alkyl
; and contacting the compound 5 with DBU or DBN in MeOH or EtOH for benzoate ester hydrolysis to yield the compound of Formula I.

Conceptually, the invention may also be summarized according to the following non-limiting chemical flow diagram:

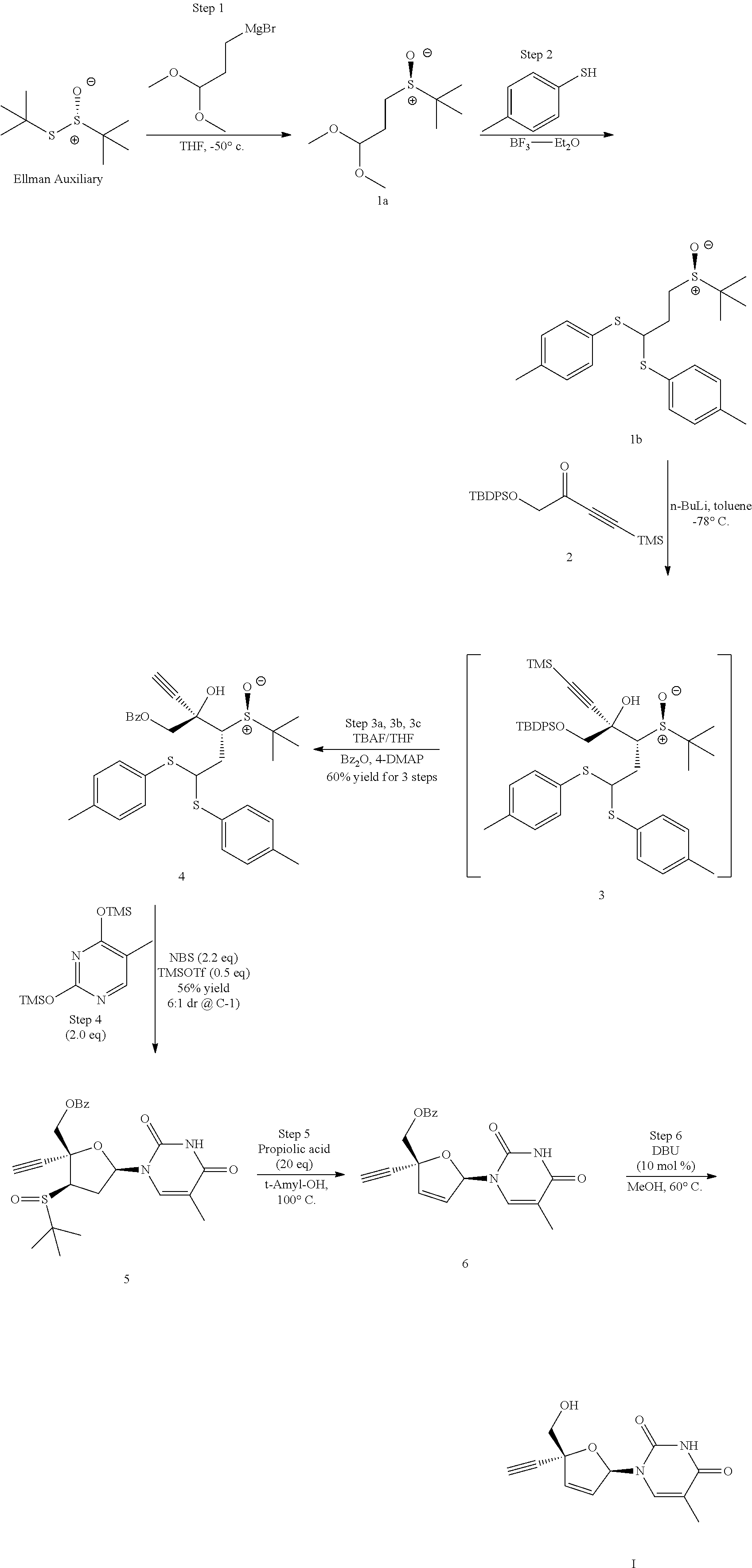

In a further embodiment, the invention is also directed to one or more of each of the individual sub-steps 1, 2, 3a-c, 4, 5, and 6 above, whether alone or in tandem.

In another embodiment of the invention, there is also provided each of the intermediate compounds 1, 2, 3, 4 and 5 above.

Also provided as part of the invention is the compound of Formula I prepared according to the process of the invention in accordance with the various embodiments herein set forth.

Yet other aspects and embodiments may be found in the description provided herein.

### DETAILED DESCRIPTION OF THE EMBODIMENTS

Unless otherwise specifically set forth, many chemical reagents have been identified herein by their commonly accepted letter abbreviations in the art for ease of reference.

### Step 1: Preparation of Compound 1a

The first step is the cryogenic Grignard addition of the commercially available 3-bromo-1,1-dimethoxy propane and the known (S)—S-tert-butyl 2-methylpropane-2-sulfinothioate. The identity of the in situ generated organometallic reagent could be comprised of either the Mg, Li, Zn, Cu, In, or Sm species. The reaction is conducted by separately generating the organometallic reagent followed by addition of the thiosulfinate as a solution in THF (tetrahydrofuran). This addition mode acts to assist in the prevention of thiosulfinate racemization. This addition results in the inversion of stereochemistry at the sulfur stereocenter, and the generation of compound 1a (75-85% yield).

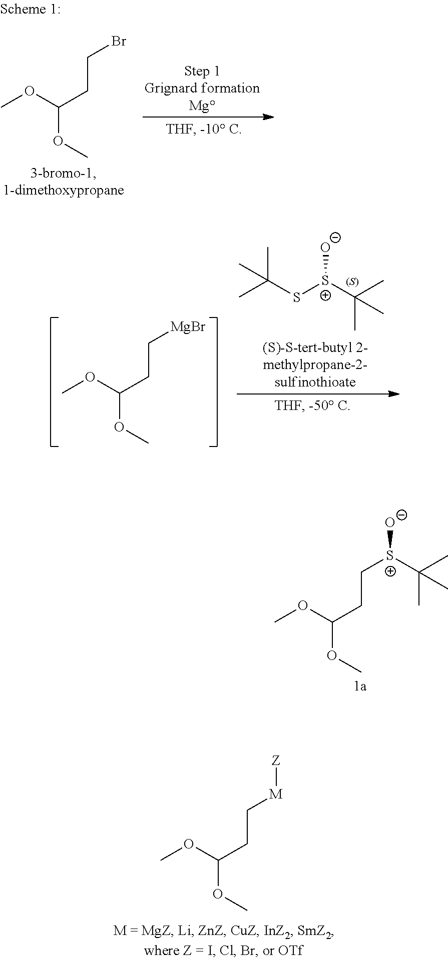

### Step 2: Preparation of Compound 1b

In this step the dimethyl acetal is converted to a dithioacetal using the Lewis acid BF₃-Et₂O in toluene or CH₂Cl₂ as solvent to generate compound 1b. The identity of the thiol component may be selected from the group of thio-alkyl, thio-cycloalky, tethered thio-alkyl, and substituted thio-aryls. The Lewis acid employed may be selected from the group of SiR₃OTf, TiCl₄, SnCl₄, and BCl₃. The reaction can also be conducted under Brönsted Acid catalysis using p-TsOH, H₂SO₄, or HCl.

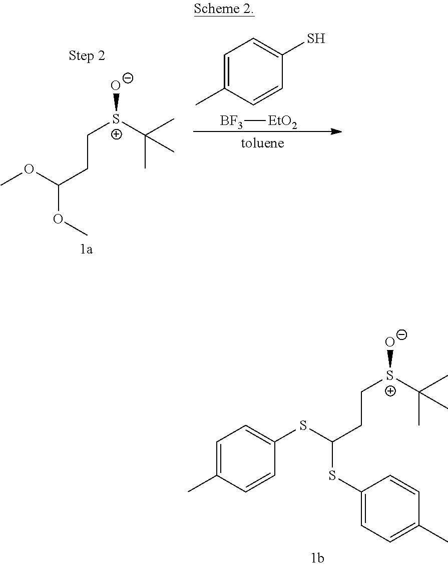

### Step 3a-c: Preparation of Compounds 3 and 4

This next step is a 3 step telescope that results in the union of compounds 2 and 1b. [3a] This transformation is complicated by the requirement of two diastereoselective events: (1) selective lithiation of the sulphoxide and (2) diastereoselective coupling of the ketone 2. Lithiation can be conducted with n-BuLi or LDA in toluene at approximately −78° C. producing the lithiated species 1c in >40:1 dr. A coordinating solvent such as THF or DME (dimethyl ether) is then added (3-5 eq). This additive provides for high reaction conversion for step 3a by promoting fragment coupling rather than proton transfer. Lithium species 1c is aged at −10° C. for 30 min, cooled to −78° C. and then a toluene solution of compound 2 is added to provides compound 3 (3:1 dr, 85-90% conversion). [3b-c] The crude mixture is treated with TBAF (tetra-n-butylammonium fluoride) in THF solvent to remove the TMS (trimethylsilyl) and TBDPS (tert-butyldiphenylsilyl) protecting groups, and then benzoylated at the C-5 hydroxyl group using benzoic anhydride and 4-DMAP (4-dimethylaminopyridine) in t-Amyl-OH as solvent to generate compound 4 (60% overall yield) as a crystalline solid.

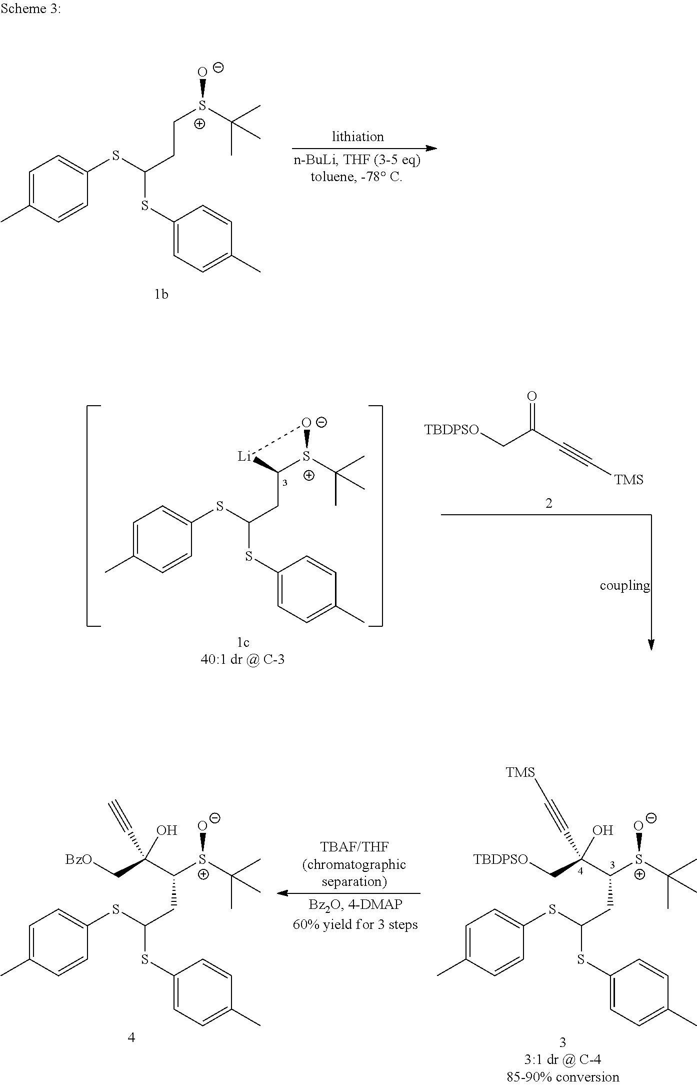

### Step 4: Preparation of Compound 5

This is a one pot two step reaction starting with the oxidation of dithioacetal 4 using NBS (N-bromosuccinimide) (2-2.5 eq) in nitromethane or acetonitrile in the presence of bis-TMS (trimethylsilyl)-thymine (1.5-2.0 eq) and TMSOTf (trimethylsilyl triflate) (0.5-1.0 eq). The initial oxidation, which could employ NCS (n-chlorosuccinimide) or NIS (n-iodosuccinimide), facilitates the generation of the 5-membered furanose ring while the second oxidation event allows for the stereoselective introduction of the thymine unit (6:1 dr) to generate compound 5 in (50-56% yield). The origin of the stereoselectivity can be traced to the C-3 sulphoxide stereocenter which appears to allow the desired “internal delivery” mode of addition. At this point in this synthetic route it is important to note that the single stereogenic sulfur atom has diastereoselectively introduced the C-1 (indirectly), C-3, and C-4 stereocenters (directly).

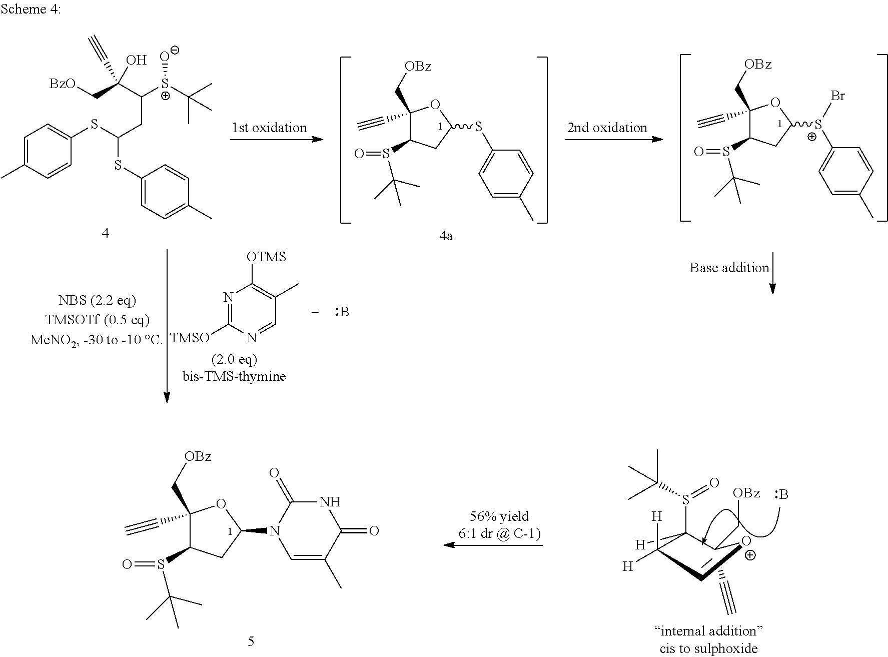

### Step 5: Preparation of Compound 6

This is the penultimate step which involves the thermal elimination of the tert-butyl sulphoxide to generate the required C2-C3 olefin present in the final compound I. Without being bound by any particular theory, this may be the first example of the use of a t-butyl sulphoxide as a masking group or handle for the installation of an olefin. The reaction involves the initial liberation of isobutylene and the generation of sulfenic acid 5a. In the absence of a propiolic acid/esters, 5a will undergo a dimerization reaction and fail to proceed to compound 6. However, in the presence of propiolic acid/esters or any irreversible conjugate acceptor, 5a can be intercepted and funneled to vinyl sulphoxide intermediate 5b, which is capable of undergoing the desired sigmatropic rearrangement and furnish unsaturated compound 6. Compound 6 can be isolated directly from the reaction mixture when the reaction is conducted in an alcoholic solvent such as n-BuOH or t-Amyl-OH.

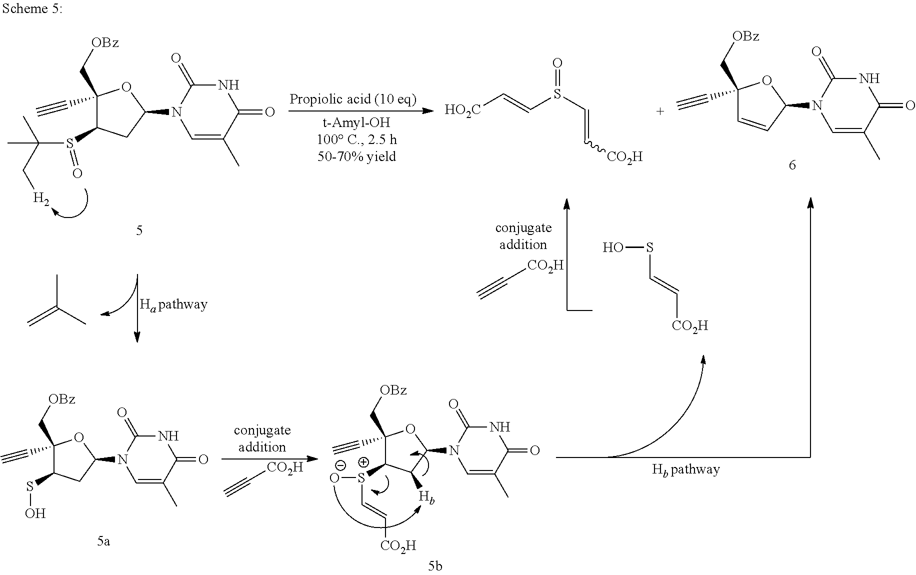

### Step 6: Preparation of Compound I

This is the API step which involves the DBU (1,8-diazabicycloundec-7-ene) catalyzed transesterification of the C-5 benzoate ester protecting group to the solvent (MeOH). This fully organic process (i.e. substantially H₂O free) eliminates the need for an aqueous work up and may be more efficient than previous processes which employed a NaOH mediated hydrolysis in aq. THF. This second generation process can be conducted using catalytic amounts (about 0.025-0.10 eq) of a variety of organic medium strength bases such as DBU, DBN (1,5-diazabicyclo(4.3.0.)non-ene), or TMG (1,1,3,3,-tetramethylguanidine) with MeOH as solvent. MeOH is an important solvent for this transformation, as the reaction does not proceed under identical conditions using high order alcohols such as EtOH, IPA (isopropyl alcohol) or n-BuOH. It is the preferred acid/base match between solvent and base. The reaction proceeds to completion within about 8-24 h (depending on catalyst loading). Solvent swap is performed into EtOH, and the compound I is isolated from EtOH/heptanes which provides for the desired form and required particle properties of the API.

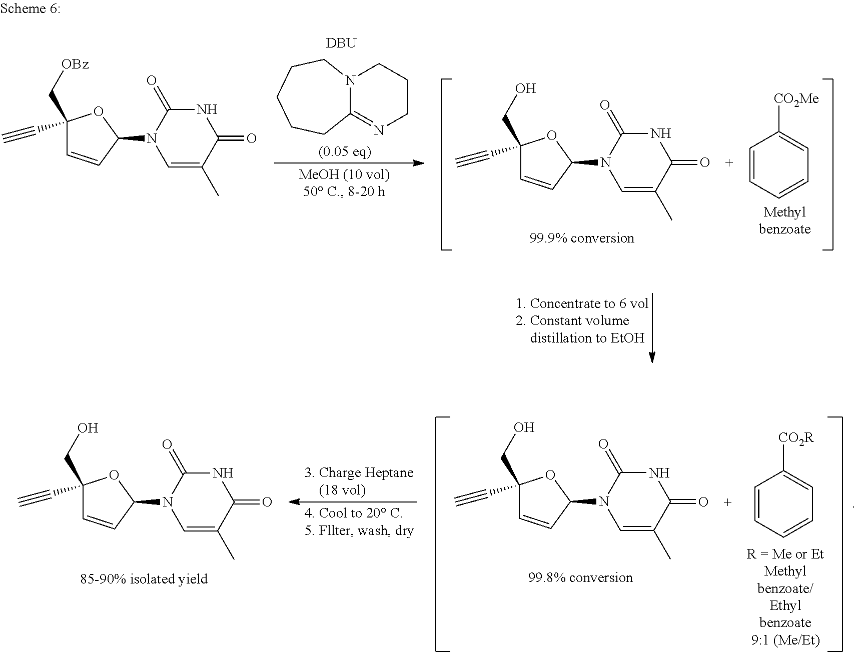

The foregoing description is merely illustrative and should not be understood to limit the scope or underlying principles of the invention in any way. Indeed, various modifications of the invention, in addition to those shown and described herein, will become apparent to those skilled in the art from the foregoing description and examples. Such modifications are also intended to fall within the scope of the appended claims.

What is claimed is:

1. A process for making the compound of Formula I 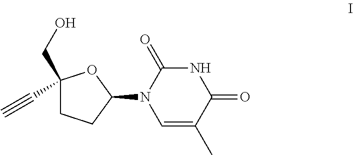 [SMILES: C#C[C@@]1(CO)CC[C@H](n2cc(C)c(=O)[nH]c2=O)O1]
which comprises:
(1) contacting the starting compound (S)—S-tert-butyl 2-methylpropane-2-sulfinothioate with the Grignard reagent generated from 3-bromo-1,1-dimethoxypropane to produce 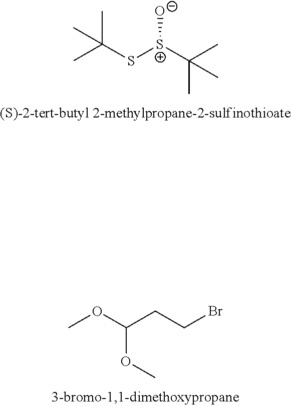
compound 1a. 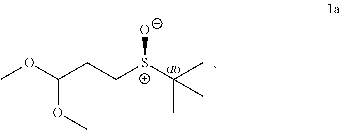
and then contacting compound 1a with p-thiocresol and BF₃-Et₂O to produce the compound 1b 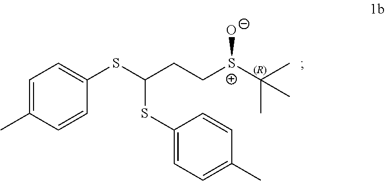
and
(2) contacting compound 1b with n-BuLi and compound 2 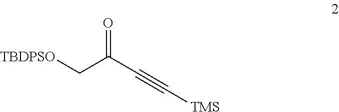
to produce the compound 3, after removal of the silicon groups with TBAF and then re-protection of the C-5 hydroxy group as its benzoate ester (4-DMAP, Bz₂O) 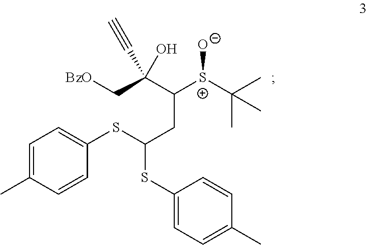
and
(3) contacting the compound 3 with Bis-TMS-thymine 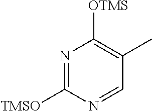
and NBS in nitromethane or CH₃CN to produce compound 4 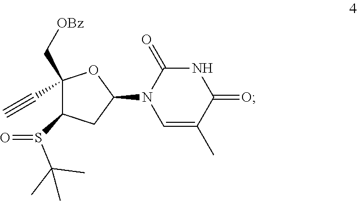
and
(4) contacting the compound 4 with propiolic acid with n-BuOH or t-Amyl-OH as solvent and heating to produce compound 5 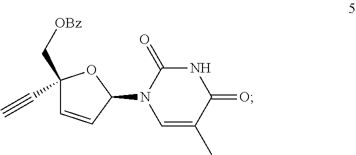
and
(5) contacting the compound 5 with DBU or DBN in MeOH or EtOH for benzoate ester hydrolysis to yield the compound of Formula I.

2. The compound of Formula I prepared according to the process of claim 1.
